(19) United States
(12) Patent Application Publication
(10) Pub. No.: US 2010/0145313 A1
(43) Pub. Date: Jun. 10, 2010

(54) SYSTEM AND METHOD FOR TREATING SEPTAL DEFECTS

(76) Inventor: Brian M. Packard, Monticello, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/581,369

(22) Filed: Oct. 19, 2009

Related U.S. Application Data
(63) Continuation-in-part of application No. 12/533,908, filed on Jul. 31, 2009.
Application No. 12/533,908 is a continuation of application No. 10/818,135, filed on Apr. 5, 2004, now abandoned.
Application No. 10/818,135 is a continuation-in-part of application No. 10/818,089, filed on Apr. 5, 2004.
(60) Provisional application No. 60/460,544, filed on Apr. 4, 2003.
(60) Provisional application No. 60/460,696, filed on Apr. 4, 2003.

(51) Int. Cl. A61M 25/16

(52) U.S. Cl. 604/535

(57) ABSTRACT

A system for introducing and advancing medical devices into vessels, including an introducer with a first elongated tubular member having a first connection element at a distal end and a guide catheter comprising a second connection element at a proximal end, the first and second elongated tubular members being substantially hollow for receiving and passing through an expandable device for treatment of septal defects, where the first connection element includes a female connection element with an internal continuous annularly shaped protruding rib; and the second connection element comprises a male connection element adapted to be inserted into the first connection element, the second connection element having an external continuous annularly shaped protruding rib, where the internal rib and external rib are configured to interlock and resist relative longitudinal movement while allowing relative rotational movement. Methods of making and using are also described as well as an associated cutting apparatus.

### CROSS-REFERENCE TO RELATED APPLICATION(S)

This application is a continuation-in-part application of, and claims priority to, the following applications: U.S. patent application Ser. No. 12/533,908, filed on Jul. 31, 2009, entitled Guide Catheter and Method of Making Same, which application is a continuation application of U.S. patent application Ser. No. 10/818,135 filed on Apr. 5, 2004, entitled Guide Catheter and Method of Making Same, which application claims priority to U.S. provisional application No. 60/460,544, filed Apr. 4, 2003; and U.S. patent application Ser. No. 10/818,089 filed on Apr. 5, 2004, entitled Introduction Apparatus, which application claims priority to U.S. provisional application No. 60/460,696, filed Apr. 4, 2003. Each of the above listed applications are hereby incorporated herein by reference in their entireties.

### FIELD OF THE INVENTION

The present invention relates to a guide catheter and a method of manufacturing the guide catheter. Further, the present invention relates to a guide catheter configured to be used with expandable devices and devices with sharp components without damage to the catheter, and a method of manufacturing such a catheter. The present invention also relates to an apparatus for introducing a medical device into a catheter. It further relates to an apparatus for introducing medical devices that are flexible or have geometries that don't facilitate introduction.

### BACKGROUND OF THE INVENTION

Guide catheters are typically used to guide instruments such as balloon catheters, guidewires or similar devices to specific locations in a human body to perform their specific function, such as angioplasty. The inner layer of such catheters range from 0.0005 inches to 0.0015 inches. One problem with current guide catheters is that they are damaged or rendered inoperable due to weakness in the materials as a result of the insertion of current expandable nickel titanium devices or devices with sharp components.

There is a need in the art for a guide catheter that has a thin wall thickness yet has the wall strength to withstand the use of expandable devices made of such materials as nickle titanium or devices with sharp components.

Catheters and other similar medical devices are typically introduced into vessels by pushing the distal end of the device forward using the catheter shaft as support. The medical devices that are introduced in this manner may include intravascular devices, expanded nitinol meshes, balloon catheters, or similar devices that require exterior support to allow for advance of the device. Further, the devices include those where loading an element from the distal end is preferred as not to damage the primary catheter tip or intravascular portion.

Often the introduction and advancement of such a device into a vessel is difficult because the device is either too flexible or has a geometry that doesn't facilitate introduction. Further, loading such a device may damage the primary tip or, in the case of an intravascular device, the intravascular portion.

As such, in addition to the guide catheter needs discussed above, there is a need in the art for an apparatus that allows for the introduction and advancement of catheters and other medical devices into vessels. There is a further need for an apparatus that allows for the protection of the primary tip or any distal portions of the device.

### BRIEF SUMMARY OF THE INVENTION

In one embodiment, a system for introducing and advancing medical devices into vessels may include a catheter with a male connection element at a proximal end, the male connection element being cylindrically shaped, surrounding the catheter, and having a proximal end extending beyond the proximal end of the catheter defining a predetermined distance. The male element may further include an external annularly shaped protruding rib at its proximal end. The system may also include an elongated tubular member with a first connection element just proximal to a distal end, the first connection element being a female connection element and having an opening at its distal end and a back wall at its proximal end, the distal end of the elongated tubular member extending beyond the back wall of the first connection element a distance substantially equal to the predetermined distance allowing for an abutting relationship between the distal end of the elongated tubular member and the proximal end of the catheter. The first connection element may further include an internal, annularly shaped, protruding rib configured to interlock with the external annularly shaped protruding rib of the male element, the external and internal protruding ribs being configured to resist relative longitudinal movement while allowing relative rotational movement.

In another embodiment, the elongated tubular member of the previous embodiment may also include a second connection element near a proximal end. In another embodiment, the second connection element may be a luer connector. In another embodiment, an inner diameter of the elongated tubular member may be substantially the same as an inner diameter of the catheter, the inner diameters and the abutting relationship providing for a smooth transition between the elongated tubular member and the catheter. In another embodiment, the elongated tubular member is substantially hollow and unobstructed. In another embodiment, the elongated tubular member is adapted to receive an expanding nitinol mesh and allow the mesh to pass smoothly there through. In another embodiment, the system may be adapted to provide continuous uninterrupted external support to the mesh as the mesh is first received by the elongated tubular member and further advanced into the catheter. In another embodiment, the elongated tubular member may further include a substantially constant circular inner and outer cross-section and a substantially hollow and unobstructed internal cylindrical volume adapted to receive a first medical device and allow the device to pass there through. In still another embodiment, the first connection element may be sleevably positioned over the elongated tubular member and securely adhered thereto. In yet another embodiment, the elongated tubular member may further include grooves in the outer surface at the location of the first connection element. In another embodiment, the first connection element further comprises a sealing element. In yet another embodiment, the sealing element may be generally tubular with a constant inner diameter and a tapering outer diameter, the outer diameter decreasing from the proximal end to the distal end, the sealing element extending distally from the back wall first connection element and being positioned concentrically with the inner cylindrical volume of the elongated tubular member.

In another embodiment, a system for introducing and advancing medical devices into vessels may include a tubular loader with an inner diameter and an outer diameter, the loader being configured to allow a medical device to pass there through and a guide catheter. The guide catheter may include a catheter portion including an inner layer with an etched outer surface, a support element attached to the etched outer surface, the support element configured to provide shape retention to the guide catheter, and a pressure applied outer layer external to the support element, the outer layer defining an outer diameter, wherein, an inner diameter of the guide catheter is adapted to receive and pass through an expandable device for the treatment of septal defects. The guide catheter may also include a connection portion adapted to receive the loader, the connection portion being positioned over a proximal end of the catheter portion and comprising a circumferential protruding rib positioned on the outer surface of the tubular shaft adjacent the proximal end of the connection portion, the rib being adapted to engage a corresponding rib on the loader to form a positive connection, where the connection allows for relative rotation of the catheter and the introducer while maintaining a positively connected condition.

In another embodiment, the connection portion of the guide catheter in the embodiment above may further include a tubular shaft with an inner and an outer surface, the inner surface defined by a first inner diameter and a second inner diameter with a transition there between, the first inner diameter being substantially equal to the outer diameter of the catheter portion and the second inner diameter being substantially equal to the outer diameter of the introducer, the proximal end of the catheter portion being positioned adjacent to the transition. In another embodiment, the support element may be a braided wire. In another embodiment, the support element may be a kinkless coil. In another embodiment, the pressure applied outer layer may attach to the inner layer through the gaps in the support element. In another embodiment, the support element may be made of tungsten. In another embodiment, the inner layer may have a thickness of from about 0.0015 inches to about 0.006 inches.

In another embodiment, a system for introducing and advancing medical devices into vessels may include an introducer comprising a first elongated tubular member with a first connection element at a distal end and a guide catheter comprising a second elongated tubular member having an inner layer, an outer layer, and a support element positioned there between, the guide catheter further comprising a second connection element at a proximal end, the first and second elongated tubular members being substantially hollow for receiving and passing through an expandable device for treatment of septal defects. The first connection element may include a female connection element with an internal continuous annularly shaped protruding rib and the second connection element may include a male connection element adapted to be inserted into the first connection element, the second connection element having an external continuous annularly shaped protruding rib, where the internal rib and external rib are configured to interlock and resist relative longitudinal movement while allowing relative rotational movement thereby maintaining a positively connected condition.

In another embodiment, the male connection element in the embodiment described above may be cylindrically shaped, surround the catheter, and further include a proximal end extend beyond the proximal end of the catheter defining a predetermined distance. In another embodiment, the male connection element may include an inner and an outer surface, the inner surface being defined by a first inner diameter and a second inner diameter with a transition there between, the first inner diameter being substantially equal to the outer diameter of the second elongated tubular member and the second inner diameter being substantially equal to the outer diameter of the first elongated tubular member, the proximal end of the second elongated tubular member being positioned adjacent to the transition. In another embodiment, the female connection element may include an opening at its distal end and a back wall at its proximal end, the distal end of the first elongated tubular member extending beyond the back wall of the female connection element a distance substantially equal to the predetermined distance allowing for an abutting relationship between the distal end of the elongated tubular member and the proximal end of the catheter. In another embodiment, the internal rib may be spaced from the back wall of the female connection element a distance substantially equal to a thickness of the external rib, both the distance and the thickness being measured parallel to a longitudinal axis of the elongated tubular members. In another embodiment, the first connection element may further include a sealing element. In another embodiment, the sealing element may be generally tubular with a constant inner diameter and a tapering outer diameter, the outer diameter decreasing from the proximal end to the distal end, the sealing element extending distally from the back wall of the female connection element and being positioned concentrically with the first elongated tubular member.

### BRIEF DESCRIPTION OF THE DRAWINGS

FIG. 1A is a side view of a guide catheter, according to one embodiment of the present invention.

FIG. 1B is a perspective view of a portion of a guide catheter, according to one embodiment of the present invention.

FIG. 1C is a side view of a catheter tip, according to one embodiment of the present invention.

FIG. 2A is a side view of a guide catheter, according to an alternative embodiment of the present invention.

FIG. 2B is a side view of a catheter tip, according to one embodiment of the present invention.

FIG. 3A is a cutaway side view of a connection element, according to one embodiment of the present invention.

FIG. 3B is a cutaway side view of a connection element mated with another connection element, according to one embodiment of the present invention.

FIG. 3C is a cutaway side view of a connection element, according to an alternative embodiment of the present invention.

FIG. 3D is a cutaway side view of a connection element mated with another connection element, according to an alternative embodiment of the present invention.

FIG. 4 is a flow chart depicting a method of manufacturing a catheter, according to one embodiment of the present invention.

FIG. 5A is a top view of a cutting apparatus, according to one embodiment of the present invention.

FIG. 5B is a side view of a cutting apparatus, according to one embodiment of the present invention.

FIG. 6 is a side view of an introduction apparatus, according to one embodiment of the present invention.

FIG. 7A is a cutaway side view of a first connection element, according to one embodiment of the present invention.

FIG. 7B is a cutaway side view of a first connection element connected to a catheter, according to one embodiment of the present invention.

FIG. 8A is a cutaway side view of a first connection element, according to an alternative embodiment of the present invention.

FIG. 8B is a cutaway side view of a first connection element connected to a catheter, according to an alternative embodiment of the present invention.

FIG. 9 is a flow chart of a method of using an introduction apparatus, according to one embodiment of the present invention.

FIG. 10 is a side view of an introduction apparatus having a tubular arm, according to one embodiment of the present invention.

FIG. 11 is a flow chart of a method of using an introduction apparatus, according to one embodiment of the present invention.

FIG. 12A is a perspective view of a portion of a tubular member, according to one embodiment of the present invention.

FIG. 12B is a flow chart of a grooving process, according to one embodiment of the present invention.

FIG. 13 is a flow chart of a method of adding external elements to a tubular member, according to one embodiment of the present invention.

### DETAILED DESCRIPTION

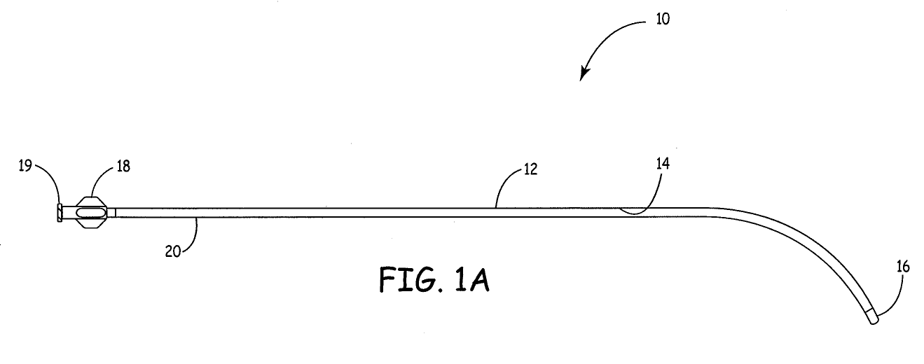

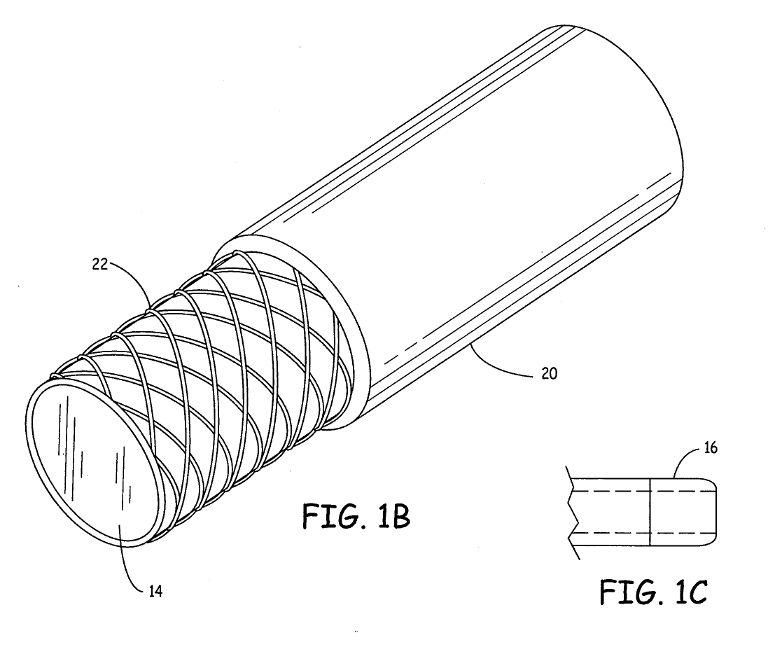

FIG. 1A depicts a guide catheter 10 according to one embodiment of the present invention. The guide catheter 10 has an elongated tubular member 12 having an inner layer 14, a catheter tip 16, a connection element 18 including a male element 19, and an outer layer 20. According to one embodiment, the guide catheter 10 is a sheath guide catheter configured to be used in conjunction with a dilator guide catheter wherein the dilator is inserted into the sheath, as will be described in further detail herein.

The inner layer 14 according to one embodiment has a thickness of from about 0.0015 inches to about 0.006 inches. The layer 14 further may be a slippery inner surface configured to promote the advancement of any device inserted into the guide catheter 10. According to one embodiment, the layer 14 is any fluoropolymer. For example, according to one embodiment the layer 14 is comprised of PTFE. Alternatively, the layer 14 is comprised of MFA. In a further alternative, the inner layer 14 is any similar low-friction material.

The thickness of the inner layer 14 provides a surface that is difficult to damage by insertion of abrasive objects or devices that apply circumferential forces. Further, the thickness of the inner layer 14 prevents the guide catheter 10 from producing unwanted debris and allows for insertion into the body vessels without creating complications.

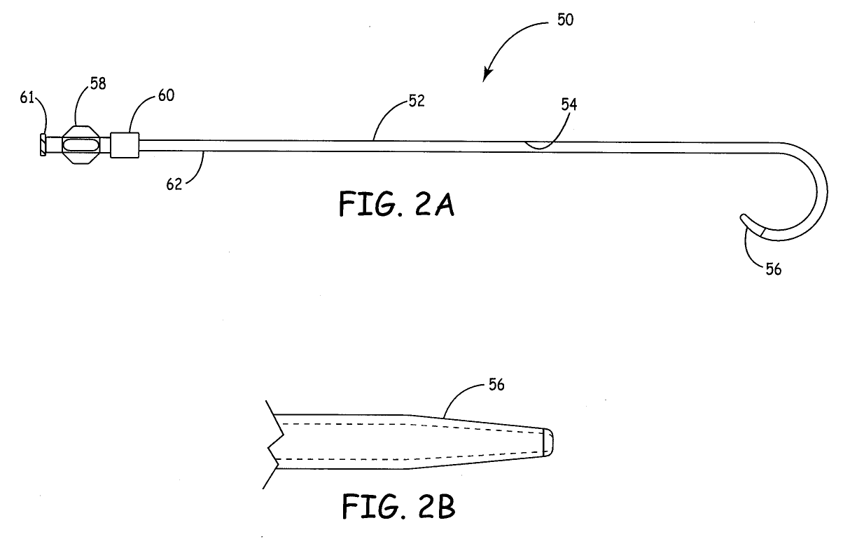

FIG. 1B depicts a portion of a guide catheter 10, according to one embodiment of the present invention. The guide catheter 10 has a support element 22 integrated into the catheter 10. According to one embodiment, the support element 22 is braided wire. Alternatively, the support element 22 is a flexible, kinkless coil. As shown in FIG. 2, the support element 22 is wrapped around an external portion of the inner layer 14. The thickness of the inner layer 14 optimizes the ability to include the support element 22. According to one embodiment, the support element 22 is comprised of tungsten.

The support element 22 is configured to provide a predetermined shape to the guide catheter 10 that can aide the operator in accessing a desired location for use. Further, according to one embodiment, the support element 22 provides a stiffness or rigidity that allows an operator to steer or direct the catheter 10 to difficult locations that require that the catheter 10 withstand resistance. For example, according to one embodiment, the catheter 10 is used to access a membranous ventricle septal defect (“membranous VSD”). According to an alternative aspect of the invention, the support element 22 provides a kink resistance to the catheter 10, such that when the catheter 10 is bent or deformed, no kink or permanent deformation results. For example, according to one embodiment, the support element 22 allows the catheter 10 to be used with a tortuous device such that the tortuous device can be inserted into or through the catheter 10 without resulting in permanent kinks or deformation to the catheter 10.

The outer layer 20 according to one embodiment is configured to be exterior to the support element 22. Further, the outer layer 20 may conform to the shape of the support element 22 and, according to one embodiment in which the support element 22 is braided wire, can be attached to the inner layer 14 in the gaps (also referred to as “pics”) between the braided wires.

The connection element 18 is associated with the tubular member 12 at the proximal end of the tubular member 12. The connection element 18 according to one embodiment is configured to receive devices. According to one embodiment, the connection element 18 has an internal diameter (“I.D.”) that matches the outer diameter (“O.D.”) of the tubular member 12. According to a further embodiment, the connection element 18 has a male element 19 configured to be coupleable with a female element on a connection device or loader. In operation, the insertion into the connection element 18 of a connection device or loader having an O.D. that is the same as the tubular member 12 allows for smooth insertion of a device through the connection device or loader and into the guide catheter 10.

FIG. 1C depicts a catheter tip 16, according to one embodiment of the present invention. The catheter tip 16 is associated with the distal end of the guide catheter 10. The tip 16 is configured to prevent portions of the support element 22 to be exposed at the end of the catheter 10.

FIG. 2A depicts a guide catheter 50, according to an alternative embodiment of the present invention. The guide catheter 50 has an elongated tubular member 52 having an inner layer 54, a catheter tip 56, a connection element 58 including a female element 60 and a male element 61, and an outer layer 62. According to one embodiment, the guide catheter 50 is a dilator guide catheter configured to be used in conjunction with a sheath guide catheter such as, for example, guide catheter 10, wherein the dilator is inserted into the sheath, as will be described in further detail herein.

The inner layer 54 and outer layer 62, according to one embodiment, have the same or substantially the same characteristics, composition, and structure as the inner layer 14 and outer layer 20, respectively, described herein. According to an alternative aspect of the invention, the guide catheter 50 has a support element (not shown) integrated into the catheter 50, wherein the support element has the same or substantially the same characteristics, composition, and structure as the support element 22 described herein. In a further alternative, the guide catheter 50 has no support element.

FIG. 2B depicts a catheter tip 56 according to one embodiment of the present invention. The catheter tip 56 is associated with the distal end of the guide catheter 50. The tip 56 is configured to prevent portions of the support element (not shown) to be exposed at the end of the catheter 50.

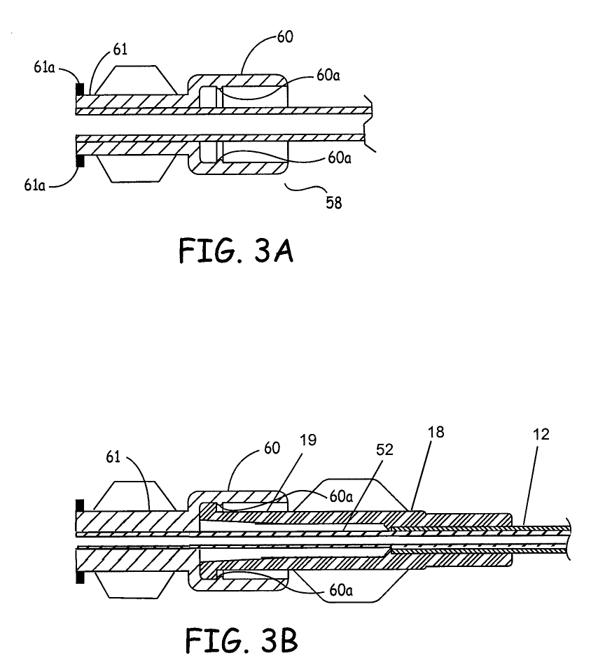

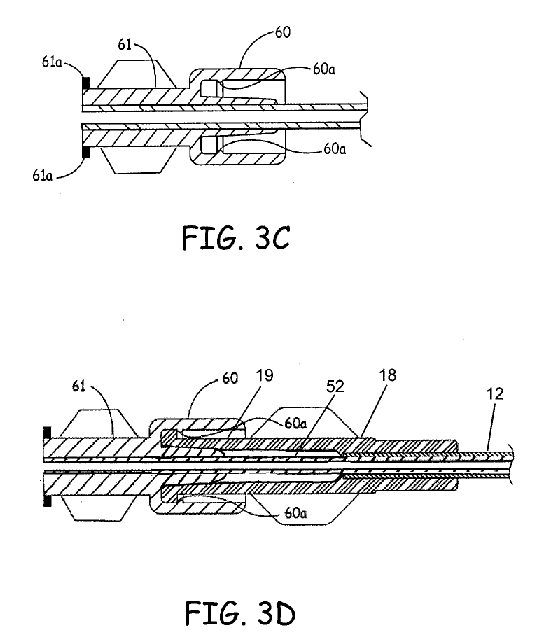

FIG. 3A depicts a connection element 58, according to one embodiment of the present invention. The connection element 58 is associated with the tubular member 52 at the proximal end of the tubular member 52 as shown in FIG. 2A. The connection element 58 according to one embodiment is configured to receive devices and further to connect to devices that the tubular member 52 is inserted into. According to one embodiment, the connection element 58 has an internal diameter (“I.D.”) that matches the outer diameter (“O.D.”) of the tubular member 52. In operation, the insertion into the connection element 58 of a connection device or loader having an O.D. that is the same as the tubular member 52 allows for smooth insertion of a device through the connection device or loader and into the guide catheter 50.

According to a further aspect of the invention, the connection element 58 as depicted in FIG. 3A has a male element 61 configured to be coupleable with a female element on a connection device or loader that is inserted into the catheter 50. The male element 61 has protruding elements 61a that are configured to contact a female element such that the male 61 and female elements are held in connection and can be separated only with some force being applied.

According to another embodiment, the connection element 58 has a female element 60 as shown in FIG. 3A configured to be coupleable with a male element on a device into which the catheter 50 is inserted. The female element 60 has inner protruding elements 60a that are configured to contact protruding elements on a male element (similar to the protruding elements 61a as shown) such that the male and female 60 elements are held in connection and can be separated only with some force being applied, as shown in FIG. 3B.

FIG. 3C depicts a connection element 58, according to an alternative embodiment of the present invention. The connection element 58 has a female element 60 with inner protruding elements 60a and a male element 61 with protruding elements 61a. FIG. 3D shows the female element 60 of FIG. 3C in connection with a male element.

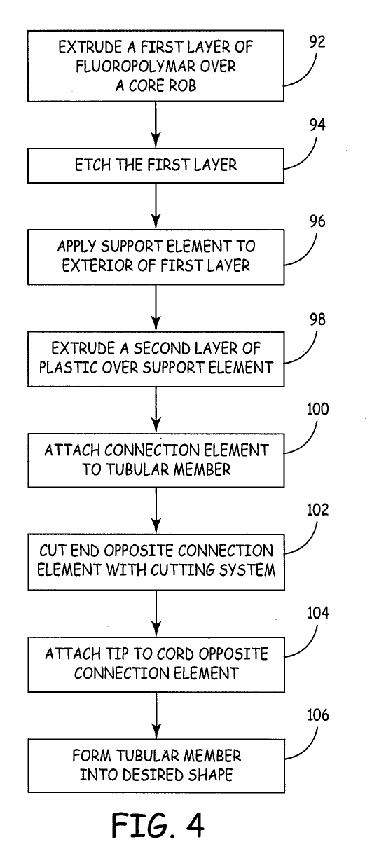

FIG. 4 depicts a method of manufacturing a catheter 90, according to one embodiment of the present invention. According to one embodiment, the method is a method of manufacturing a sheath guide catheter. Alternatively, the method is a method of manufacturing a dilator guide catheter. First, a first layer of a fluoropolymer is extruded onto a core rod (block 92). In an alternative aspect of the invention, the extruded material can be any known extrudable polymer. According to one embodiment the core rod is copper. A copper rod can be stretched after the fluoropolymer has been extruded onto, thereby causing the diameter of the rod to decrease and simplifying the removal of the rod from the formed first layer. Alternatively, the core rod is plastic. According to one embodiment, this first layer will be the inner layer 14, 54 of the catheter 10, 50.

The extruded layer is then etched to create a surface to which other objects can be attached (block 94). According to one embodiment, the etching takes place by applying a sodium-based solution to the layer. In an alternative aspect of the invention, the extruded layer is not etched. Next, according to one embodiment, the support element 22 is applied to the exterior of the layer (block 96). According to one embodiment, the support element 22 is applied by braiding the layer with wires. The layer may be braided with from about 8 to about 32 wires. Alternatively, the support element 22 is a kink-resistant flexible coil that is applied to the exterior of the layer. In an alternative embodiment, no support element is applied. For example, the manufacture of some dilator guide catheters does not require application of a support element.

A second layer of plastic is then extruded over the support element 22 (block 98), or if there is no support layer, the second layer is extruded over the first layer. According to one embodiment, the plastic is nylon. Alternatively, the plastic can be any known plastic for use in medical devices. According to one embodiment, air pressure is applied during this step to ensure that the second layer extends through gaps in the support element 22 and attaches to the first layer.

Once the tubular member has been manufactured, additional components can be added to create the catheter. The connection element 18, 58 is attached to an end of the tubular member 12, 52 (block 100). According to one embodiment, the connection element 18, 58 is attached by molding the connection element 18, 58 onto the end of the tubular member 12, 52. That is, an appropriate mold is placed on the end of the tubular member 12, 52 and hot liquid material is added to the mold such that the material forms a connection element 18, 58 that is molded to the end of the tubular member 12, 52. According to one embodiment, the molding step is accomplished with a molding machine. Alternatively, the connection element 18, 58 is attached by any known means for attaching a component to a catheter.

In one alternative embodiment, the end of the tubular member 12, 52 is cut with a cutting system (block 102) prior to attachment of a tip 16, 56. For some embodiments, cutting the end serves to expose an end of the tubular member and facilitate attachment of a tip. In a further alternative, cutting the end of a tubular member having a support member exposes the support member as well, thereby facilitating complete encapsulation of the support member with the tip. According to one embodiment, a mandrel is inserted into the tubular member 12, 52 prior to the cutting step to facilitate cutting by providing support to the tubular member 12, 52 during the process. In a further embodiment, the cutting system used is a two-blade cutting system described in further detail below.

A tip 16, 56 is then attached to the end of the tubular member 12, 52 opposite the connection element 18, 58 (block 104). According to one embodiment, the tip is formed from an existing portion of the end of the tubular member 12, 52. The end is heated by the application of radio frequency (“R.F.”) energy and then shaped appropriately. Alternatively, the tip is formed by a molding step in which an appropriate piece of plastic is heated, molded into the appropriate shape, and formed onto the catheter using R.F. energy. According to one embodiment, the R.F. energy is applied using R.F. dies.

In accordance with one alternative aspect of the present invention, the resulting catheter 10, 50 is then formed into a desired shape. That is, the catheter 10, 50 is placed in hot liquid to make the catheter moldable. Alternatively, the catheter 10, 50 may be placed on heated platens to make it moldable. The catheter 10, 50 can then be formed into the desired shape. Subsequently, the catheter 10, 50 is placed in cold liquid to eliminate its moldability.

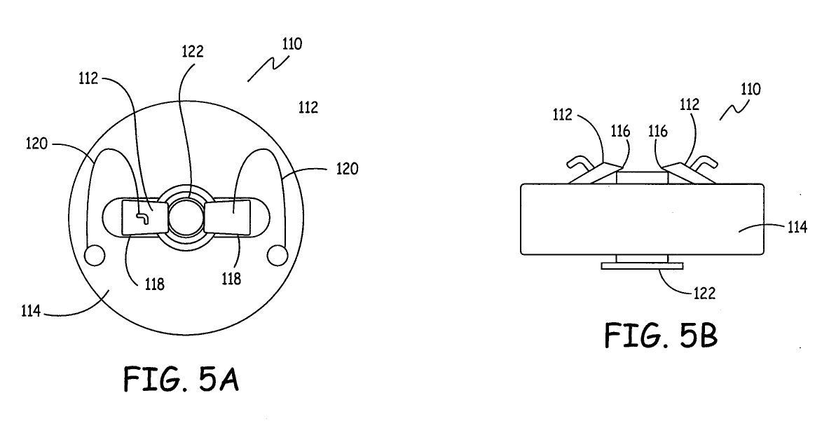

FIG. 5A depicts a top view of a cutting system 110, according to one embodiment of the invention. FIG. 5B depicts a side view of a cutting system 110, according to one embodiment of the invention. According to one embodiment, the cutting system 110 can be used to cut the tubular member 12, 52 as described above. The cutting system 110 has two blades 112 with cutting edges 116. The blades 112 are pivotably coupled to a base 114 with pivot rods 118 inserted through holes at the non-cutting end of the blades. The cutting system 110 also has tension wires 120 connected at one end to the base 114 and at the other end to the blades 112. The tension wires 120 provide a tension urging the cutting edges 116 of the blades 112 toward the base 114. Alternatively, the cutting system 110 can have any known component configured to urge the blades 112 toward the base 114 or provide a downward force or tension on the blades 112 toward the base 114.

The system 110 has a positioning element 122 moveably disposed in the center of the base 114 and in contact with both blades 112. According to one embodiment, the positioning element 122 is a tube element. The tube element 122 is configured to move the blades 112 between a cutting position and a non-cutting position. That is, when the tube 122 is urged upward (toward the blades side of the base 114), the blades 112 are urged upwards and the distance between the cutting edges 116 increases. When the upward force on the tube 122 is removed, the downward force of the tension wires 120 urges the blades 112 downward and the distance between the cutting edges 116 decreases. Alternatively, the positioning element 122 can be any component configured to move the blades 112 between a non-cutting position and a cutting position. The base 114 is configured to rotate or spin around the tube element 122 such that a tubular member 12, 52 disposed within the tube element 122 can be cut by the two blades 112.

According to one embodiment, the two blades 112 cut at two different locations around the circumference of the tubular member 12, 52, thus applying an equal amount of pressure around the circumference and cutting in a precise manner that prevents exposure of any portion of the support element 22 by forcing the support element 22 inward as it cuts. Alternatively, the cutting system 110 can have three blades 112. In a further alternative, the cutting system 110 can have 1 to 4 blades 112.

In operation, the cutting system 110 can be used to cut a tubular member 10, 50. First, the tube element 122 is urged upward, thereby urging the blades 112 upward and increasing the distance between them. When the tube element 122 has urged the blades 112 upward such that the distance between the blades 112 is greater than the O.D. of the tubular member 12, 52 to be cut, the tubular member 12, 52 is inserted through the tube element 122. Once the tubular member 12, 52 is properly positioned, the force on the tube element 122 is released and the blades 112 are urged downward and closer together by the tension wires 120 until they are in contact with the tubular member 12, 52. Then the base 114 is caused to rotate or spin such that the blades 112 spin around the tubular member 12, 52, thereby cutting the tubular member 12, 52.

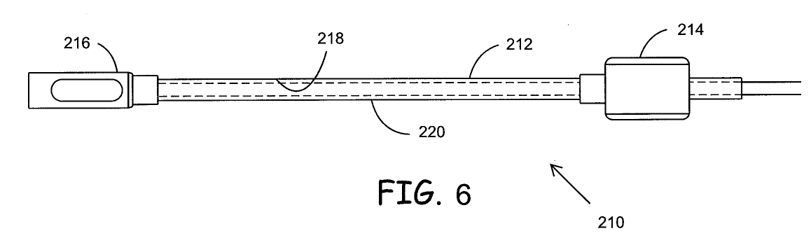

Turning now to FIG. 6, an introduction apparatus 210 is shown according to one embodiment of the invention. The introduction apparatus has an elongated tubular member 212 and a first connection element 214 and a second connection element 216.

The first connection element 214, according to one embodiment, is associated with the elongated tubular member 212 at a distal portion of the tubular member 212. According to one embodiment, the first connection element 214 is located just proximal to the distal end of the tubular member. The first connection element 214 is configured to be attachable to a catheter. According to one embodiment, the first connection element 214 is a lure lock. Alternatively, the first connection element 214 is a snap or press fit. In a further alternative, the first connection element 214 can be any known connection device.

The location of the first connection element 214 just proximal to the distal end of the tubular member 212 allows the elongated tube to extend into the catheter and provide a relatively seamless introduction of a medical device into the catheter. In a further embodiment, the first connection element 214 is located anywhere in the distal portion of the tubular member 212. Alternatively, the first connection element 214 is at the distal end of the tubular member.

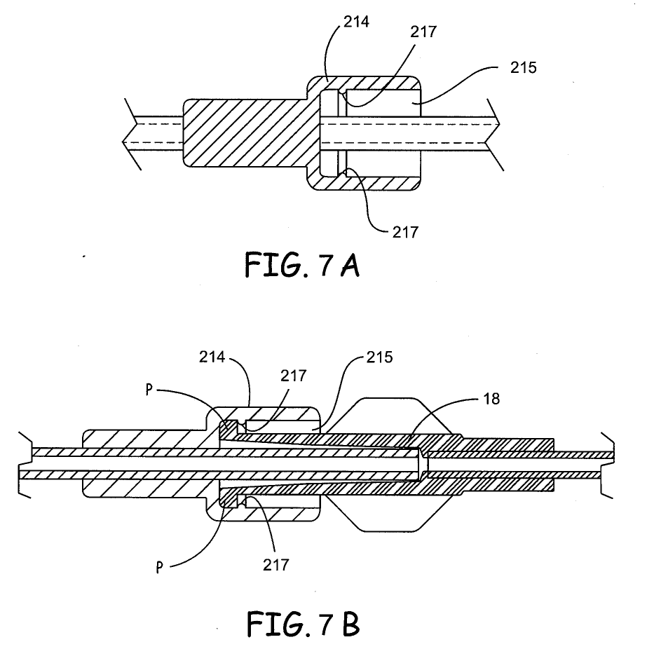

FIG. 7A depicts a cutaway side view of a first connection element 214, according to one embodiment of the present invention. FIG. 7B depicts a cutaway side view of the first connection element 214 of FIG. 7A in mated connection with a male portion 18 of a catheter, according to one embodiment of the present invention. The connection element 214, according to one embodiment, is a “female” connection element having an opening 215 at one end configured to receive an appropriate “male” connection portion 18 of a catheter. Inside the opening 215, the connection element 214 has two small protruding elements 217. The protruding elements 217 are configured to contact protruding elements P on the male element 18 such that the male element 18 and the first connection element 214 are held in mated connection and can be separated only with some force being applied.

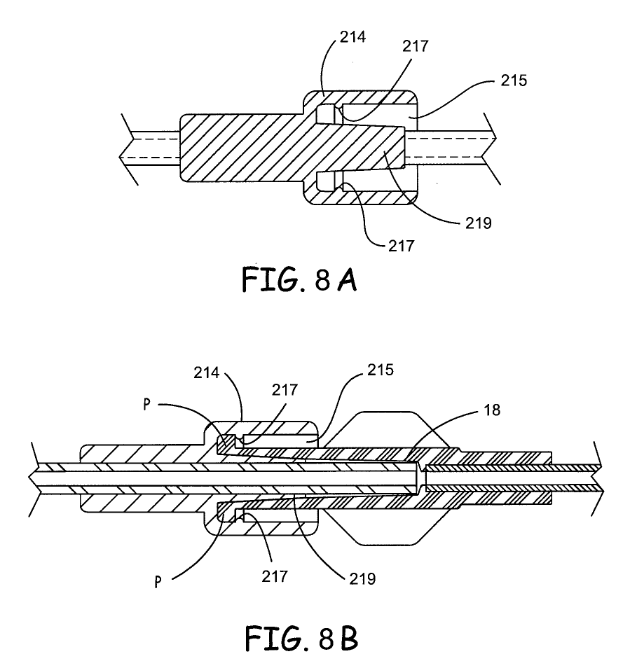

FIG. 8A depicts a cutaway side view of a first connection element 214, according to an alternative embodiment of the present invention. FIG. 8B depicts a cutaway side view of the first connection element 214 of FIG. 8A in mated connection with a male portion 18 of a catheter, according to one embodiment of the present invention. The connection element 217, according to one embodiment, is a “female” connection element having an opening 215 at one end configured to receive an appropriate “male” connection portion 18 of a catheter. Inside the opening 215, the connection element 214 has two small protruding elements 217 and a sealing element 219. The protruding elements 217 are configured to contact protruding elements P on the male element 18 such that the male element 18 and the first connection element 214 are held in mated connection and can be separated only with some force being applied. The sealing element 219 is configured to provide a tighter mating connection or “seal” between the first connection element 214 and the male element 18 by contacting and maintaining contact with an inner portion of the male element 18.

Returning to FIG. 6, the second connection element 216, according to one embodiment, is associated with the elongated tubular member 212 at the proximal end of the tubular member 212. The second connection element 216, according to one embodiment, is a lure connector. Alternatively, the second connection element is any known connection element. The second connection element 16 may be used to evacuate air and flush the loader with fluids. Alternatively, the second connection element 216 is used to attach tools such as compression devices or catheters. In a further alternative, the second connection element 216 is used both to evacuate air and flush fluids and to attach tools.

The second connection element 216 can vary in size. According to one embodiment, the second connection element 216 accepts devices of sizes varying from about size 3 French to about size 16 French. Alternatively, the second connection element 216 accepts devices of sizes varying from about size 5 French to about size 12 French. In a further alternative, the second connection element 216 accepts adapters that are configured to accept devices of French sizes that are not compatible with the second connection element 216 itself.

According to one embodiment, the apparatus 210 is any fluoropolymer. For example, according to one embodiment the apparatus 210 is comprised of PTFE. Alternatively, the apparatus 210 is comprised of MFA. Alternatively, the apparatus is a co-extruded material having an inner liner 218 where the inner liner 218 is any fluoropolymer. In a further alternative, the inner liner 218 is any similar low-friction material.

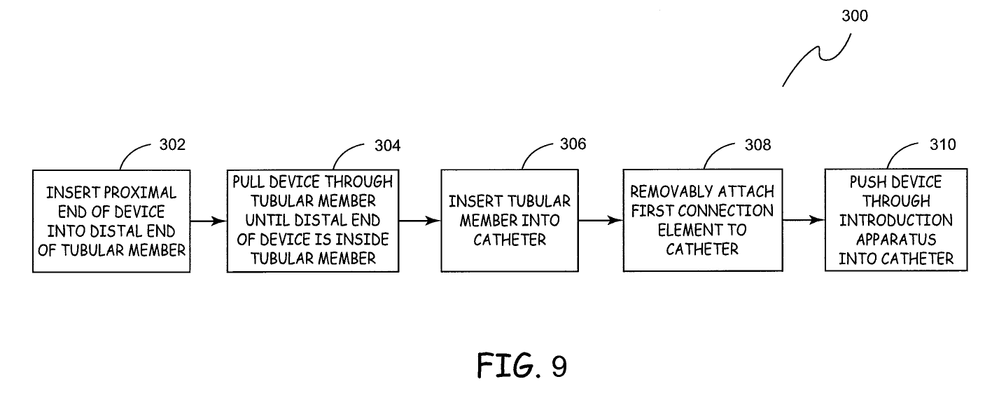

FIG. 9 depicts a method of using an introduction apparatus 300 according to one embodiment of the present invention. In operation, the proximal end of a device is inserted into the distal end of the tubular member 212 (block 302). Once the device has been pulled through the tubular member 212 such that the distal end of the device is enclosed within the tubular member 212 (block 304), the tubular member 212 is inserted into a catheter (block 306). Upon insertion, the first connection element 214 removably attaches to the catheter (block 308). At this point, the device is pushed through the introduction apparatus 210 and into the catheter to which the apparatus 210 is attached (block 310).

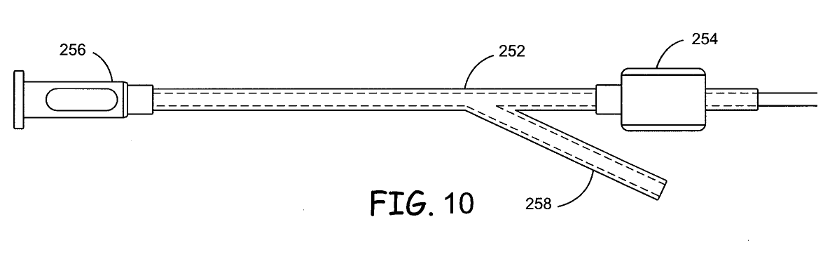

FIG. 10 depicts an introduction apparatus 250 according to an alternative embodiment of the present invention. The apparatus 250 has an elongated tubular member 252 and a first connection element 254 and a second connection element 256. The apparatus also has an additional tubular arm 258 associated with the tubular member. According to one embodiment, the tubular arm 258 is configured to accept a device for insertion into a catheter while the apparatus 250 is already attached to the catheter. In a further embodiment, the introduction apparatus 250 has an elongated tubular member 252, a tubular arm 258, and a second connection element 256 and is permanently attached at the distal end of the tubular member 252 to the catheter.

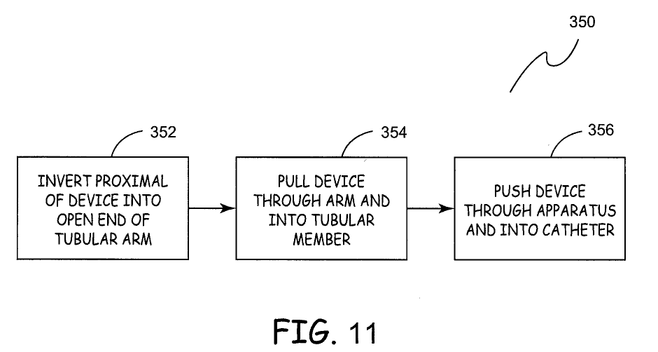

FIG. 11 depicts a method of using an introduction apparatus (250) 350 with a tubular arm 258 according to one embodiment of the present invention where the apparatus 250 is already attached to the catheter. In operation, the proximal end of a device is inserted into the open end of the tubular arm 258 (block 352). Once the device has been pulled through the tubular arm 258 and into the tubular member 252 such that the distal end of the device is enclosed within the tubular member 252 (block 354), the device is pushed through the introduction apparatus 250 and into the catheter to which the apparatus 250 is attached (block 356).

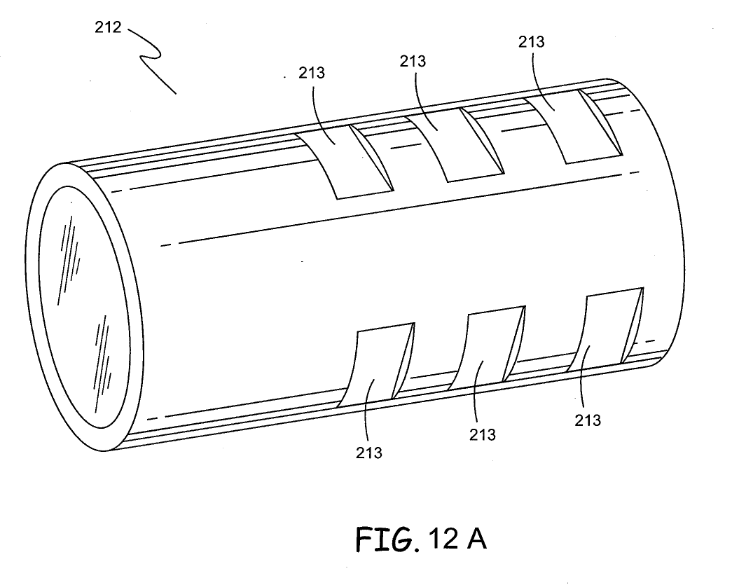

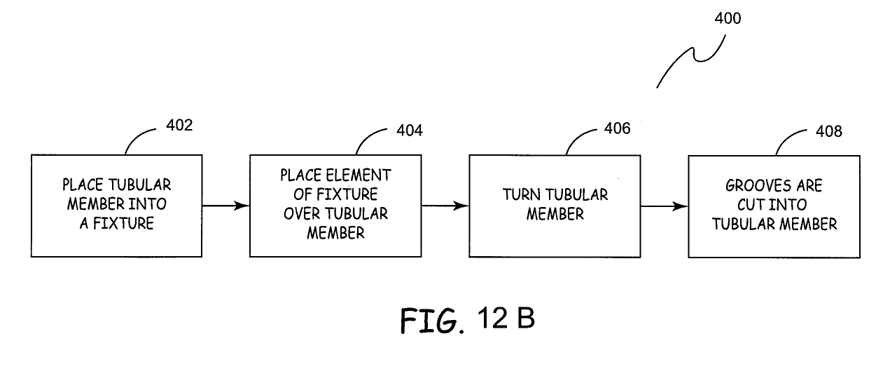

The tubular member 212 according to one embodiment is made by a known extrusion process. FIG. 12A depicts a portion of a tubular member 212 made by a process that results in a configuration that allows adherence of external elements, according to an alternative embodiment of the present invention. That is, a grooving process, which is also known as a “roughing” process, is used to create grooves 213 in the tubular member 212 which are configured to allow for the attachment of external elements, such as the first connection element 214 or the second connection element 216.

FIG. 12B depicts a grooving process 400 used to make grooves in the tubular member 212, according to one embodiment of the present invention. The tubular member 212 is placed into a fixture (block 402). Upon placement in the fixture, an element of the fixture is placed over the tubular member 212 such that grinding wheels contact the tubular member 212 at desired locations (block 404). Next, the tubular member is turned (block 406), thus causing grooves to be cut into the tubular member (block 408).

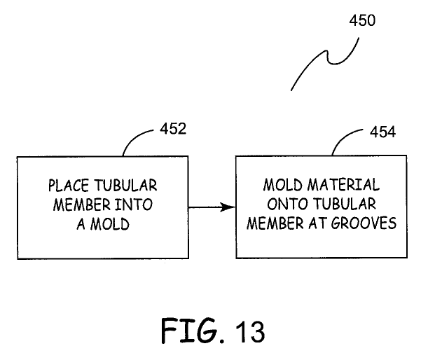

FIG. 13 depicts a method of adding external elements to the tubular member (12) 450, according to one embodiment of the invention. For example, the first connection element 214 and the second connection element 216 can be added by this process. The tubular member 212, having grooves at appropriate locations, is placed into a mold (block 452). Then an appropriate material is molded onto the tubular member 212 at the grooves (block 454).

In a further alternative, if the apparatus 210 was created using a co-extrusion process, the tubular member 212 may not need grooving if the outer layer 220 is configured to allow adherence of external elements.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

What is claimed is:

1. A system for introducing and advancing medical devices into vessels comprising:
a catheter with a male connection element at a proximal end, the male connection element being cylindrically shaped, surrounding the catheter, and having a proximal end extending beyond the proximal end of the catheter defining a predetermined distance, the male element further comprising an external annularly shaped protruding rib at its proximal end; and
an elongated tubular member with a first connection element just proximal to a distal end, the first connection element being a female connection element and having an opening at its distal end and a back wall at its proximal end, the distal end of the elongated tubular member extending beyond the back wall of the first connection element a distance substantially equal to the predetermined distance allowing for an abutting relationship between the distal end of the elongated tubular member and the proximal end of the catheter, the first connection element further comprising an internal, annularly shaped, protruding rib configured to interlock with the external annularly shaped protruding rib of the male element, the external and internal protruding ribs being configured to resist relative longitudinal movement while allowing relative rotational movement.

2. The system of claim 1, wherein the elongated tubular member further comprises a second connection element near a proximal end.

3. The system of claim 2, wherein the second connection element is a luer connector.

4. The system of claim 3, wherein an inner diameter of the elongated tubular member is substantially the same as an inner diameter of the catheter, the inner diameters and the abutting relationship providing for a smooth transition between the elongated tubular member and the catheter.

5. The system of claim 4, wherein the elongated tubular member is substantially hollow and unobstructed.

6. The system of claim 5, wherein the elongated tubular member is adapted to receive an expanding nitinol mesh and allow the mesh to pass smoothly there through.

7. The system of claim 6 adapted to provide continuous uninterrupted external support to the mesh as the mesh is first received by the elongated tubular member and further advanced into the catheter.

8. The system of claim 1, wherein the elongated tubular member further comprises a substantially constant circular inner and outer cross-section and a substantially hollow and unobstructed internal cylindrical volume adapted to receive a first medical device and allow the device to pass there through.

9. The system of claim 8, wherein the first connection element is sleevably positioned over the elongated tubular member and is securely adhered thereto.

10. The system of claim 9, wherein the elongated tubular member further comprises grooves in the outer surface at the location of the first connection element.

11. The system of claim 10, wherein the first connection element further comprises a sealing element.

12. The system of claim 11, wherein the sealing element is generally tubular with a constant inner diameter and a tapering outer diameter, the outer diameter decreasing from the proximal end to the distal end, the sealing element extending distally from the back wall first connection element and being positioned concentrically with the inner cylindrical volume of the elongated tubular member.

13. A system for introducing and advancing medical devices into vessels, comprising:
a tubular loader with an inner diameter and an outer diameter, the loader being configured to allow a medical device to pass there through; and
a guide catheter comprising:
a catheter portion including:
an inner layer with an etched outer surface;
a support element attached to the etched outer surface, the support element configured to provide shape retention to the guide catheter; and
a pressure applied outer layer external to the support element, the outer layer defining an outer diameter,
wherein, an inner diameter of the guide catheter is adapted to receive and pass through an expandable device for the treatment of septal defects; and
a connection portion adapted to receive the loader, the connection portion being positioned over a proximal end of the catheter portion and comprising a circumferential protruding rib positioned on the outer surface of the tubular shaft adjacent the proximal end of the connection portion, the rib being adapted to engage a corresponding rib on the loader to form a positive connection, where the connection allows for relative rotation of the catheter and the introducer while maintaining a positively connected condition.

14. The system of claim 13, the connection portion of the guide catheter further comprising a tubular shaft with an inner and an outer surface, the inner surface defined by a first inner diameter and a second inner diameter with a transition there between, the first inner diameter being substantially equal to the outer diameter of the catheter portion and the second inner diameter being substantially equal to the outer diameter of the introducer, the proximal end of the catheter portion being positioned adjacent to the transition.

15. The system of claim 14, wherein the support element is a braided wire.

16. The system of claim 15, wherein the support element is a kinkless coil.

17. The system of claim 16, wherein the pressure applied outer layer attaches to the inner layer through the gaps in the support element.

18. The system of claim 17, wherein the support element is made of tungsten.

19. The system of claim 18, wherein the inner layer has a thickness of from about 0.0015 inches to about 0.006 inches.

20. A system for introducing and advancing medical devices into vessels, comprising:
an introducer comprising a first elongated tubular member with a first connection element at a distal end; and
a guide catheter comprising a second elongated tubular member having an inner layer, an outer layer, and a support element positioned there between, the guide catheter further comprising a second connection element at a proximal end,
the first and second elongated tubular members being substantially hollow for receiving and passing through an expandable device for treatment of septal defects;
wherein:
the first connection element comprises a female connection element with an internal continuous annularly shaped protruding rib; and
the second connection element comprises a male connection element adapted to be inserted into the first connection element, the second connection element having an external continuous annularly shaped protruding rib,
wherein, the internal rib and external rib are configured to interlock and resist relative longitudinal movement while allowing relative rotational movement thereby maintaining a positively connected condition.

21. The system of claim 20, wherein the male connection element is cylindrically shaped, surrounds the catheter, and further comprises a proximal end extending beyond the proximal end of the catheter defining a predetermined distance.

22. The system of claim 21, wherein the male connection element includes an inner and an outer surface, the inner surface defined by a first inner diameter and a second inner diameter with a transition there between, the first inner diameter being substantially equal to the outer diameter of the second elongated tubular member and the second inner diameter being substantially equal to the outer diameter of the first elongated tubular member, the proximal end of the second elongated tubular member being positioned adjacent to the transition.

23. The system of claim 21, wherein the female connection element comprises an opening at its distal end and a back wall at its proximal end, the distal end of the first elongated tubular member extending beyond the back wall of the female connection element a distance substantially equal to the predetermined distance allowing for an abutting relationship between the distal end of the elongated tubular member and the proximal end of the catheter.

24. The system of claim 23, wherein the internal rib is spaced from the back wall of the female connection element a distance substantially equal to a thickness of the external rib, both the distance and the thickness being measured parallel to a longitudinal axis of the elongated tubular members.

25. The system of claim 23, wherein the first connection element further comprises a sealing element.

26. The system of claim 25, wherein the sealing element is generally tubular with a constant inner diameter and a tapering outer diameter, the outer diameter decreasing from the proximal end to the distal end, the sealing element extending distally from the back wall of the female connection element and being positioned concentrically with the first elongated tubular member.
